(19) United States
(12) Patent Application Publication
(10) Pub. No.: US 2013/0066334 A1
(43) Pub. Date: Mar. 14, 2013

(54) Axial Surgical Trajectory Guide

(71) Applicant: Hans Schoepp, Freiburg (DE)

(72) Inventor: Hans Schoepp, Freiburg (DE)

(73) Assignee: Stryker Leibinger GmbH & Co., KG, Freiburg (DE)

(21) Appl. No.: 13/618,673

(22) Filed: Sep. 14, 2012

Related U.S. Application Data
(63) Continuation-in-part of application No. 13/228,083, filed on Sep. 8, 2011.

(51) Int. Cl. A61B 19/00

(52) U.S. Cl. 606/130

(57) ABSTRACT

A medical trajectory guide includes a base a configured for alignment with a coordinate axis, a first guide member rotatably attached to the base, a guide sleeve guided by the first guide member, and a motor coupled to the first guide member and configured to rotate the first guide member in relation to the first axis. The first guide member is rotatable about a first axis.

### CROSS REFERENCE TO RELATED APPLICATIONS

This application is a Continuation-In-Part and claims the benefit of U.S. non-provisional application Ser. No. 13/228,083, filed Sep. 8, 2011, the contents of which are hereby incorporated by reference.

### BACKGROUND OF THE INVENTION

1. Field of the Invention

The present invention relates to apparatus, and methods related to guiding an axial medical instrument, such as a needle or probe, during a medical procedure.

2. Description of the Background of the Invention

During medical procedures it is often necessary for a surgeon to insert an axial surgical instrument, such as a needle, shunt, or probe, into a patient to reach a pre-selected target within the body of the patient. Some exemplary surgical procedures where this necessary include biopsy procedures and invasive neurosurgical procedures. In order to obtain relatively accurate placement of the axial surgical instrument, it is currently customary to use navigation techniques that rely on one or more images of the patient that include the target, such as well known computed tomography (CT) techniques, which provide three-dimensional volume image information of the patients body (i.e., a “CT image” or “CT data set”). As used herein, the term CT is not limited to a particular scanning technology, and encompasses any available technology, such as X-ray, 2D projection X-ray, C arm devices, magnetic resonance imaging, ultrasound, and other types of imaging devices capable of producing an image or a volume slice of the patient's body usable for planning and performing a navigated surgical procedure.

After a CT image is obtained of the patient, the target and an entry point are identified in the CT image by the medical personnel and from these points a linear trajectory for the surgical instrument is identified. This is often performed with the assistance of computer system controlled with appropriate software, which will calculate a pitch angle and a yaw angle with respect to a vertical gravity vector and a coordinate system relative to the CT scanner. The entry point is marked on the patient by known mapping techniques, such as with reference to a grid or other marker visible on both the patient and in the CT image. From the pitch and yaw angles, the trajectory may then be transferred from the image space to the actual patient space, and the surgical instrument can be aligned with respect to the entry point.

Thus, for example, in interventional radiology percutaneous needle placement under CT guidance is a state of the art procedure. The patient is positioned in a gantry of a CT scanner and several CT scans are obtained at different times during the procedure to localize the entry point and insert the needle to the target point in the body, Some exemplary applications include soft tissue biopsies (e.g. lung, liver, kidney), bone biopsies, vertebroplasty, RF ablation, etc. The procedure is typically performed with a straight needle-shape instrument. Localization of the entry point is generally less problematic than localization of the target point inside the patient. For entry point localization, the patient is typically scanned with a grid in the area of interest. The grid stripes are identified in the CT scan and are used to correlate the planned entry point as identified in the CT image with the physical entry point on the patient. For target point localization the physician usually has to align the angle manually according to the plan from the CT image and needs regular periodic scanning to monitor and control the trajectory. Thus guidance of the needle to the target is an iterative process with iterative control scans and often requires a revision of the needle trajectory path if the target is missed. This makes the procedure time consuming and yields a high amount of X-ray exposure to the patient and physician. Modern CT scanners provide a “CT-Fluoro” mode which allows constant imaging in a few slices. This feature allows the physician to visualize live the penetration of the needle into the tissue, but it also exposes the patient and staff to a high dosage of X-ray and allows only approaches in the axial plane of the CT (“in plane approach”), Often, however, an approach to the target in the plane of the CT scan is not possible, such as when a rib blocks the approach or when critical soft tissue would have to be penetrated.

Other known methods for aligning the surgical instrument in the patient space in the same orientation as planned in the CT image space are often cumbersome and/or require complex navigation systems.

Some systems use a surgical navigation system to help the surgical personnel guide a needle. For example, U.S. Patent Application Publication No. 2008/0200798, discloses a biopsy needle guide that has two independent arcuate angle guides to allow a guide tube to be pivoted about a single point and that is adapted to be adhesively attached to the skin of the patient. The needle guide is specially adapted to be visible in the CT scan image and also has specially adapted navigation markers that are tracked by a separate optical computer surgical navigation system in a manner known in the art. Images from the surgical navigation system are then registered with the CT scan images to allow the surgical staff to ensure that the needle guide is aligned on the patient in the orientation as defined in the CT scan image. In one embodiment, the biopsy needle guide is specially designed with spaced apart markers to help identify proper alignment with the CT scan image plane. Another needle guidance system based on an optical navigation system is disclosed in U.S. Pat. No. 7,876,942, in which an optical navigation camera is attached directly to a biopsy needle and a patch with fiducial markers is attached to the patient in the region of the selected entry point on the patient. Such systems, however, require complex optical surgical navigation systems in addition to CT imaging apparatus and usually require the attachment of tracking markers on the patient in order to be able to register the CT image with the patient.

In other systems, a biopsy needle is aligned in relation to the local vertical gravity vector without the use of an optical computer surgical navigation system. Some representative systems that operate on this principle are disclosed by U.S. Patent Application Publication No. 2005/0033315; EP 0 414 130; and EP 0 535 378. In these systems, generally, a bubble level or other mechanical leveling device is used to and maintain an alignment guide in a level plane, i.e., perpendicular to the local vertical gravity vector, so that a needle may be inserted at some defined trajectory angle while keeping the needle guide level. A drawback of these systems, however, is that the alignment guides must generally be maintained in the level condition and aligned in the plane of a perpendicular CT scan image plane in order to be able to accurately define the desired trajectory.

EP 1 977 704 discloses an alignment guide that uses pendulums to automatically identify the gravity vector in two, perpendicular planes so that the needle guide may be angularly adjusted in two independent, perpendicular planes. In this manner, the needle is adjusted about a yaw angle (transverse horizontal axis) by one set of pendulums, and has a protractor for adjusting the needle entry angle about the pitch angle (longitudinal horizontal axis). The alignment guide is designed to be held freehand by an operator and not to be attached to the patient. Again, a drawback of this system is that the alignment guide must be maintained at a particular level orientation in at least one degree of rotational freedom.

The inventors of the present invention have developed a system that can overcome at least some of the drawbacks of the previously known systems by eliminating the need for an external optical navigation system or the need to maintain the axial guide in particular the level plane before being able to calculate the desired trajectory in the patient space.

### SUMMARY OF THE INVENTION

In one embodiment of the present invention, a medical trajectory guide is provided. The medical trajectory guide includes a base configured for alignment with a coordinate axis. The guide further includes a first guide member rotatably attached to the base. The first guide member is configured to be rotatable about a first axis. The guide sleeve is guided by the first guide member, and a motor is coupled to the first guide member to rotate the first guide member in relation to the first axis.

According to a second embodiment of the present invention, a medical trajectory guide comprises a base configured for alignment with a coordinate axis of an imaging device. The medical trajectory guide further includes a first drive member support, a guide sleeve, and a first drive member. The first drive member support includes a first end and a second end. The first end of the first drive member support is rotatably attached to the base and the second end includes an aperture. The guide sleeve includes a first end and a second end. The first end of the guide sleeve is in contact with the base. The first drive member is linearly movable through the aperture of the first drive member support and attached to the second end of the guide sleeve such that movement of the first drive member causes the guide sleeve to tilt. A motor is configured to move the first drive member through the aperture of the first drive member support.

According to a third embodiment of the present invention, a medical trajectory guide comprises a base configured for alignment with a coordinate axis, and a first guide member rotatably attached to the base. The first guide member is configured to be rotatable about a first axis, The guide sleeve is guided by the first guide member, and a motor is coupled to the first guide member to rotate the first guide member in relation to the first axis. A controller is in communication with the motor and includes a switch having a first position, The motor is configured to rotate the first guide member about the first axis according to input from the controller if and only if the switch is in the first position.

Other aspects and advantages of the present invention will become apparent upon consideration of the following detailed description,

### BRIEF DESCRIPTION OF THE DRAWINGS

FIG. 1 is a schematic view of a patient operatively positioned in a CT scanner showing the basic parts of the CT scanner and coordinate frames;

FIG. 2A is an isometric view of an embodiment of a trajectory guide 200 according to the invention having an angular adjustment mechanism including two orthogonally aligned arcuate angular guides;

FIG. 2B is an isometric view of the trajectory guide 200 placed on a patient;

FIG. 2C is an isometric view of the trajectory guide 200 with a pitch angle of α;

FIG. 2D is an isometric view of the trajectory guide 200 with a pitch angle of α, and a yaw angle of β;

FIG. 3A is an elevational view of an alternative trajectory guide 300;

FIG. 3B is an isometric view of the trajectory guide 300;

FIG. 3C is an isometric view of the trajectory guide 300 with locking clamps 336 being compressed by an operator in order to adjust angular guide 322;

FIG. 4A is an upper isometric view of an alternative trajectory guide 400 at a neutral orientation;

FIG. 4B is an upper isometric view of the trajectory guide 400 with the guide sleeve 404 at a pitch angle of α;

FIG. 4C is an upper isometric view of the trajectory guide 400 with the guide sleeve at a pitch angle of α and a yaw angle of β;

FIG. 5 is an isometric view of an alternative trajectory guide 500; and

FIG. 6 is an isometric view of an alternative trajectory guide 600.

### DETAILED DESCRIPTION

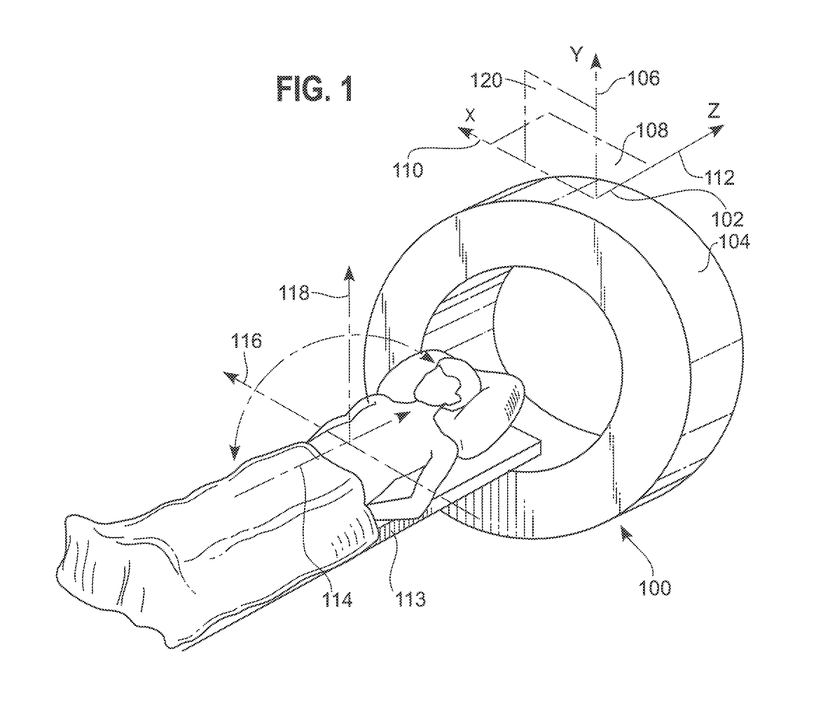

When conducting a surgical procedure, such as a biopsy, with the assistance of a computed topographic (CT) guidance system, some basic assumptions may often be made about the setup of the CT scanner that can aid in navigation of surgical tools, such as a biopsy needle, for example. As shown in FIG. 1, a CT scanner 100 customarily has a coordinate system 102, or CT coordinate space, associated with a gantry 104 for use in navigation and other spatial calculations that may be required for beneficial use of the CT scanner 100 and associated CT image data (not shown). The coordinate system 102 includes an X axis 110, a Y axis 106, and a Z axis 112. A typical CT scanner has a laser that marks a line parallel to the X-axis 110 and located on an X-Y plane 120 of the CT scanner 100. It is customary to set up the CT scanner gantry 104 in a “level” orientation, such that the vertical Y-axis 106 is parallel to a local gravity vector (not shown). When the CT scanner gantry 104 is level the vertical Y-axis is perpendicular to an X-Z plane 108 formed by the X-axis 110 and the Z-axis 112 of the CT coordinate space. A patient couch 113 is typically mounted on the gantry 104 of the CT scanner 100 in a “level” orientation parallel to the Z-axis 112 and perpendicular to the Y-axis 106.

One aspect of the disclosed system relates to guiding a needle-like instrument in an interventional radiology procedure under CT guidance. As used herein, “CT” refers to any and all methods of providing 2D or 3D image data for medical procedures, such as X-ray tomography, magnetic resonance (MR) guidance, or other similar image modality devices that produce dimensional image data. A CT scanner identifies and marks an entry point on a patient's body in a conventional way, such as by grid on the patient that is visible in CT images obtained by the CT scanner. After a first CT scan (diagnostic scan) is acquired on a CT console computer (not shown), an entry point and a target point is determined. A trajectory from the entry point to the target point is then defined on the computer and preferably shown on a display screen. With the entry point, target point, and trajectory identified, the CT console computer can compute and display the angle, relative to gravity, that the needle-like instrument must be positioned in order to enter the patient at the identified entry point and reach the identified target point, For positioning the needle-like instrument at the patient the CT scanner projects a laser line onto the patient directed in an x or z-direction of the CT coordinate system. The needle-like instrument is aligned along the laser line on the patient such that the coordinate system of the instrument coincides with the CT coordinate system (and with the patient coordinate system). Then the target point can be accurately contacted by the needle by setting only two angles of the instrument. An important requirement is that the needle-like instrument (guiding sleeve) must be prevented from rotating around the y-axis. Such a rotation would undo the alignment procedure described above. Of course, other computers not on or directly connected to the CT scanner may be used, and the term console computer is meant to encompass any such computer.

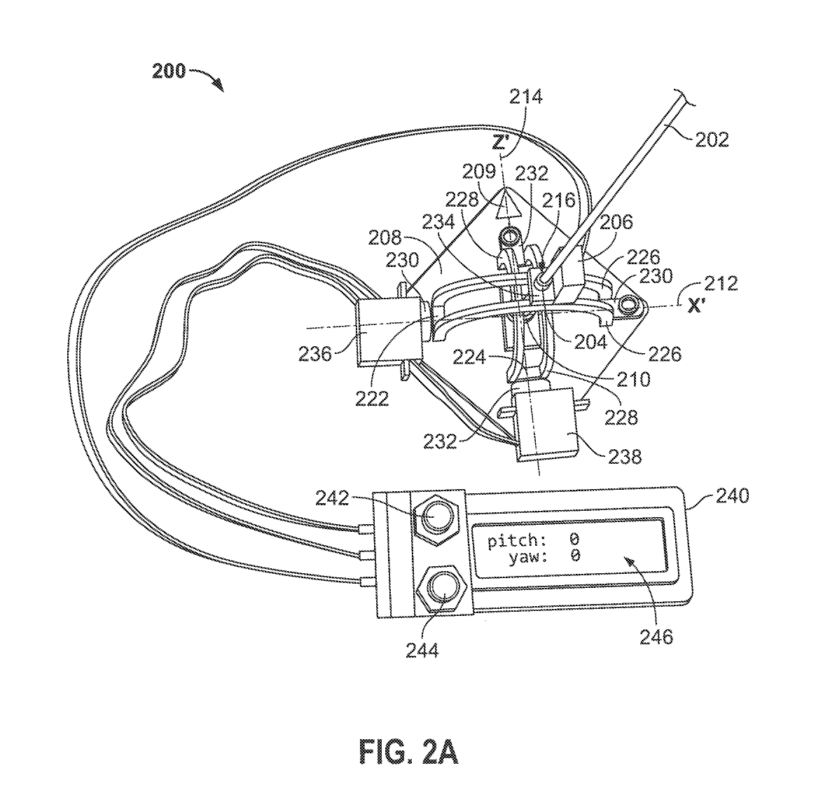

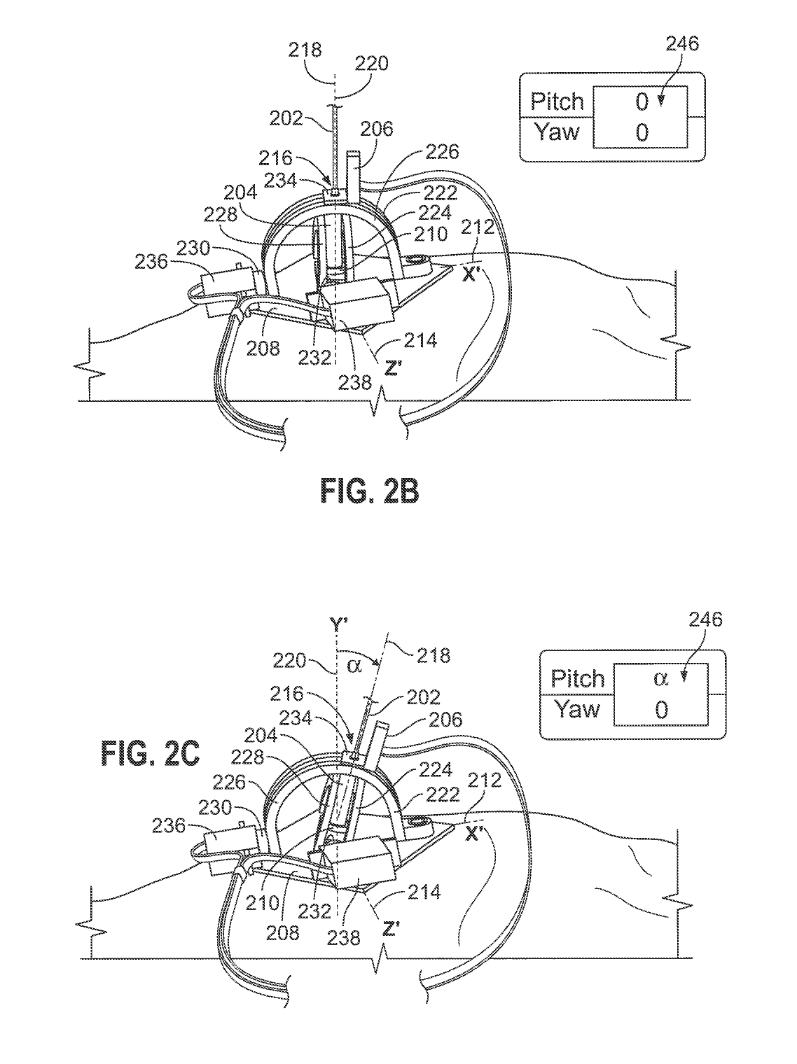

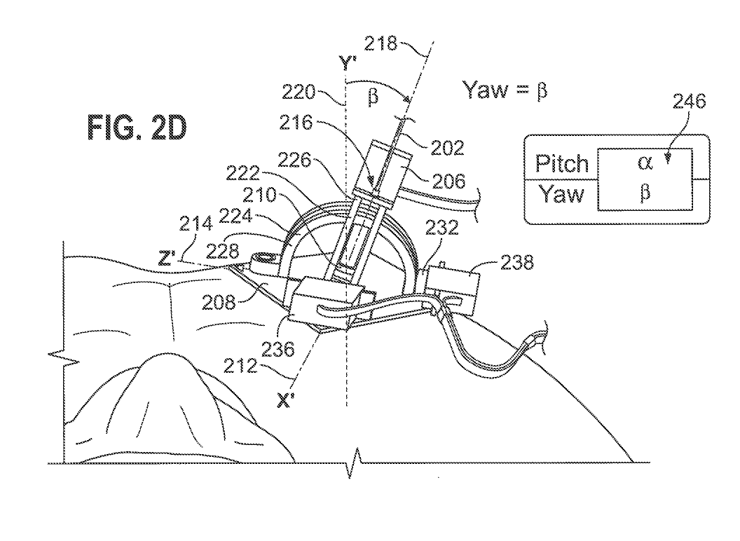

As shown in FIGS. 2A-2D, one embodiment of the present invention includes a trajectory guide 200 for positioning a needle-like instrument 202 at an appropriate angle such that it can he guided from an entry point along a trajectory to a target point. The trajectory guide 200 includes an axial guiding sleeve 204 that has an integrated angle sensor 206, such as a sensor based on micro-chip MEMS (micro electrical mechanical sensor) technology or an accelerometer. The integrated angle sensor 206 measures the angle of the guiding sleeve 204 with respect to the local gravity vector. The guide sleeve 204 is connected to a base 208 that is adapted to be placed against the patient's skin at the entry point. The guiding sleeve 204 is in the form of an elongate hollow open-ended tube and is attached to the base 208 in a manner that allows the guiding sleeve 204 to move relative to the base. The connection between the base 208 and the guiding sleeve 204 can be any suitable joint, connection, and/or attachment mechanism such as, but not limited to, a ball-and-socket joint (not shown). The guiding sleeve 204 is configured to pivot independently about two degrees of freedom through a pivot point 210.

Reference may be made to a trajectory guide 200 coordinate system that includes an X′-axis 212 and a Z′-axis 214, which extend perpendicular to one another along the base 208 and through the pivot point 210, The base 208 may include one or more markers 209 aligned with one or both of the X′-axis 212 and Z′-axis 214 of the trajectory guide 200. The pivot point 210 is disposed at or near the middle of the base 208 where the X′-axis 212 and the Z′-axis 214 intersect, The pivot point 210 is in a fixed position with respect to the base 208 and is designed to be placed directly on top of the entry point on the patient such that the pivot point 210 can be substantially coterminous with the entry point during use of the trajectory guide 200, The base 208 is preferably a flat planar member, such as a plate, frame, or ring; nevertheless, the base may be other shapes capable of being placed against the patient and preferably maintaining the trajectory guide 200 in a stable aligned position. The base 208 has an aperture (not visible) therethrough and encompassing the pivot point 210. The sleeve 204 has an aperture 210 that is sized to allow selected surgical tools, such as a biopsy needle 202, probe, or other axial device, to extend through the sleeve, through the base 208 aperture, through the entry point, and into the patient.

In alternative embodiments the guide sleeve 204 may remain unattached to the base 208. In such an alternative, the guide sleeve 204 may remain aligned with the base 208 aperture so as to guide an instrument through the aperture and along the predetermined trajectory to the target point, However, the sleeve 204 may not extend all the way down to the base. Nevertheless, persons of ordinary skill will recognize that the guide sleeve 204 need not directly engage with the base 208 to accomplish its purpose.

The sleeve 204 has a longitudinal axis 218 that can be adjusted in two degrees of freedom by angular pivoting about the X′-axis (i.e., pitch) and angular pivoting about the Z′-axis (i.e., yaw) as depicted in the drawings. When the trajectory guide 200 is at rest, the longitudinal axis 218 of the sleeve 204 is parallel to a Y′ axis 220 of the trajectory guide 200. The angle sensor 206 automatically measures the angle of the longitudinal axis 218 of the trajectory guide 200 relative to the local gravity vector and outputs digital angular data representative of the measurement values. The angle sensor 206 in this embodiment is integrated into the guiding sleeve 204 of the trajectory guide 200. In other embodiments, the angle sensor 206 may be separately attached to the guiding sleeve 204, or he integrated into a carriage member 252, or otherwise connected to the trajectory guide 200, Because the angle sensor 206 measures the angle relative to the local gravity vector, it is not necessary that the base 208 be level. By measuring the angle of the longitudinal axis 218 relative to the local gravity vector and by preventing rotation of the guiding sleeve 204 about the longitudinal axis 218, the angle of the guiding sleeve 204 with respect to the patient can be determined and the guiding sleeve 204 can be aligned with the planned trajectory.

The angle sensor 206 preferably digitally senses the change of the angle of the sensor 206 with respect to a local gravity such that a single sensor can detect changes in both the pitch and yaw of the guiding sleeve 204. The trajectory guide 200 has an appropriate data interface to transfer data from the sensor 206 to a console computer, controller, display, or some other appropriate device. The data interface may be a data port for wires or a wireless transmitter or transceiver.

In order to use a single sensor 206 to determine the pitch and yaw of the guiding sleeve 204 and to maintain alignment with the CT coordinate system, it is necessary to inhibit rotational movement of the guiding sleeve 204 about its longitudinal axis 218. This may be accomplished in many different ways, a few of which are described in relation to various embodiments disclosed herein. This allows the angle sensor 206, for example, to differentiate between movement around the X-axis 110 of the CT scanner and movement around the Z-axis 112 of the CT scanner. With this restraint on rotation of the guiding sleeve 204 about its longitudinal axis 218, calibration (or registration) of the trajectory guide is not needed. The angle sensor 206 is always referencing gravity, and is thus automatically in registration with the Y-axis 106 of the CT scanner. A simple ball-and-socket joint would not be sufficient without adding additional calibration or registration steps if rotational movement of the guiding sleeve 204 is not prevented.

The trajectory guide 200 also includes an angular adjustment mechanism comprising an outer angular guide 222 and an inner angular guide 224 disposed orthogonally to each other and aligned in the X′-Y′ and Z′-Y′ planes, respectively. Each of angular guides 222 and 224 is in the form of a pair of parallel spaced apart semi-circular rails 226 and 228 that are pivotably attached to the base 208 with two pivot connections 230 and 232 that are aligned with the respective X′ or Z′-axes. The rails 228 of the inner angular guide 224 are disposed radially inwardly from the rails 226 of the outer angular guide 222 such that the inner and outer angular guides can slide independently across each other. A carriage member 234 is connected to the guiding sleeve 204, The carriage member 234 is slidably guided by each of the angular guides 222 and 224. The carriage member 234 slides independently between and along each of the inner and outer angular guides 222 and 224 such that the guiding sleeve 204 pivots independently in pitch and yaw about the intersection of the X′-axis and the Z′-axis, which defines the pivot point 210 of the guiding sleeve 204. Thus, the longitudinal axis 218 of the guiding sleeve 204 is always aligned through the pivot point, and a needle or other axial instrument that is inserted into the aperture 216 of the alignment guide 200 will always be aligned to project through the guiding sleeve 204 into the entry point when the pivot point 210 is placed on top of the entry point on the patient. The carriage member 234 is preferably fixedly secured with the guiding sleeve 204 and prevents the guiding sleeve 204 from rotating about its own longitudinal axis 212,

While the trajectory guide 200 can be manually adjusted by an operator, preferably rotation of the angular guides 222, 224 is automated by servo motors 236 and 238, respectively. In the embodiment depicted in FIGS. 2A-2D, servo motors 236 and 238 are coupled to angular guides 222 and 224, respectively. The motors 236, 238 may be coupled to angular guides 222, 224 at their pivot connections 248 and 250 or at some other appropriate location, The motors 236 and 238 are electrically powered and configured to rotate the angular guides 222 and 224 about their respective axes without manual intervention. The motors 236, 238 are configured to be in electrical communication with a controller 240, such as through wires or a wireless transmitter or transceiver. The controller 240 may be used by an operator to manipulate the motors 236, 238, which then in turn manipulate the angular guides 222, 224, By removing the need for an operator to manually adjust or manipulate the angular guides 222, 224, and instead allowing remote manipulation through a controller 240, an operator may limit exposure to harmful radiation.

The controller 240 includes input means for adjusting the angular guides 236 and 238. As shown in FIG. 2A, the input means may include control knobs 242 and 244. Control knob 242 controls the pitch angle of the guide sleeve 204, By turning the knob 242, an operator can indicate a particular angle value a for the guide sleeve 204. When an operator adjusts the control knob 242, the controller sends an electrical signal to the motor 236, indicating that motor 236 should rotate angular guide 222 to the indicated angle α. Likewise, control knob 244 controls the yaw angle of the guide sleeve 204. By turning the knob 244, an operator can indicate a particular angle value 13 for the guide sleeve 204. When an operator adjusts the control knob 244, the controller sends an electrical signal to the motor 238, indicating that motor 238 should rotate angular guide 224 to the indicated angle β. Alternative input means may include an alphanumeric keyboard interface, arrow keys, a touchscreen, voice recognition, a joystick, a mouse, a digital file, and/or any other suitable method known to those of ordinary skill. The controller 240 may also be integrated with a computer interface in a personal computer, remote terminal, portable computer, or mobile electronic device, or any other suitable electronic device able to receive appropriate input and effectively communicate that input to the motors.

The controller 240 includes a display screen 246 that shows the pitch angle and yaw angle of the angular guides 236 and 238. As discussed above, the angles are measured relative to a local gravity vector by means of the angle sensor 206. The displayed pitch and yaw angles correspond to the angles measured by the angle sensor 206 relative to the local gravity vector. In the depicted embodiment, the controller display 246 only shows one value for each angle. This is because the user interface is much simpler and intuitive if the user is confronted with a single value only. The servo motors 236, 238 move the angular guides 222, 224 fast enough that the actual angle values quickly follow the inputted angle values, Nothing more than a small latency is noticeable by the operator.

The controller also includes a microprocessor. The microprocessor may translate the inputted values into electrical signals appropriate for communication to the motors 236, 238. Additionally, the microprocessor may handle encoding and decoding of communication signals to and from the motors 236, 238 and sensor 206. The controller 240 may include appropriate software to assist and/or enable the function of the microprocessor.

The controller 240, motors 236, 238 and angle sensor 206 cooperate to maintain the angular guides 222, 224 (and thereby the guide sleeve 204) at a desired angle. As noted above, angle sensor 206 measures the pitch and yaw of the angular guides 222, 224 relative to gravity. The local gravity vector generally does not change. In contrast, the positioning of the patient may change, for any number of reasons, and/or the positioning of the trajectory guide 200 on the patient may change. If, for example, the trajectory guide 200 shifts or otherwise changes position, the angle of the guide sleeve 204 relative to gravity may change as well. In such a case, the angle sensor 206 senses the change in angle and transmits the new angle value to the controller 240. The controller 240 recognizes that the sensed angle and the previously inputted angle value (or 0 if no angle value has been entered) are not the same and sends a signal to the appropriate motor in order to bring the sensed angle and the inputted angle into congruence.

Alternative embodiments may display an actual angle value in addition to an inputted angle value for each of the pitch and yaw angles. Additionally, the controller 240 may include means for indicating that the motors 236, 238 should begin execution, such as a button or switch (not shown). In such an embodiment, the angular guides 222, 224 are not moved by the motors 236, 238 until the execution means is activated. After the execution means is activated, the motors adjust the angular guides 222, 224 until the actual angles measured by the angle sensor 206 corresponded to the angle values inputted by the operator. Further, the controller 240 may include means for disabling and/or locking the motors, the angular guides 222, 224, and/or the input mechanism(s) entirely in order to prohibit unwanted movements of the angular guides 222, 224 during critical periods, such as when necessary for safe insertion of a medical instrument into the body.

In addition the guiding sleeve 204 can either be easily replaced by a guiding sleeve with a different diameter and/or shape to accommodate a different instrument or can be fitted with inserts to accommodate different shaped instruments.

The controller 240 can use the angular data received from the trajectory guide 200 to calculate the trajectory of the guiding sleeve 204 with respect to the Y-axis 106 of the CT scanner in the pitch and yaw degrees of freedom, i.e. gravity or local vertical, and display a representation of the trajectory in a usable manner on the display device, such as being overlaid on the CT image or other visualization useful for assisting the operator to place the guide sleeve in a desired trajectory.

In operation, an operator, such as a surgeon or radiologist, positions the trajectory guide 200 with the pivot point 210 directly on top of the selected entry point marked on the patient. The operator may align the trajectory guide 200 such that the coordinate axes X′ 212 and Z′ 214 of the trajectory guide 200 and/or one or more markers 209 are aligned with the X-axis 110 and Z-axis 112 of the gantry plane of the CT scanner 100. Preferably, one or more of the reference marks 209 on the trajectory guide 200 are aligned with a laser beam not shown) from the CT scanner 100 which projects a line on the patient that also lies on the axial plane 120 of the scanner 100 as is common for most CT scanners. The trajectory guide 200 may be fixed in position to the patient in this aligned state, such as with adhesive stickers (not shown) on the bottom of the trajectory guide 200. Of course other attachment mechanisms, such as straps or hook-and-loop fasteners may be used, Alternatively, the trajectory guide 200 may not include a fastener for affixing the base to the patient, and the operator could hold the trajectory guide in the aligned state. In any event, maintaining the trajectory guide 200 in the aligned state on the patient also allows the patient to be moved out of the CT gantry 104 on the patient couch 113 and allows the procedure to be performed outside of the CT gantry provided the orientation of the patient does not change relative to the coordinate system of the CT scanner 100. Rotational pre-alignment of the trajectory guide with the X-axis 110 or Z-axis 112 of the CT scanner 100 together with the fact that the patient couch 113 has a known alignment with respect to gravity, i.e., the Y-axis 106, realizes an angular registration between the CT coordinate system 102 and the patient on the patient couch 113 which obviates the need for additional registration between the coordinate system of the CT scanner 100 and CT image data and the coordinate system of the patient.

After the trajectory guide has been aligned with the X-axis 110, the operator inserts a needle or some other instrument 202 into the guiding sleeve 204 of the trajectory guide 200. At this point, the operator may further change the angular positions of the needle 202 via the trajectory guide 200 to match them with or modify target angles.

Once the guiding sleeve 204 is aligned with the planned trajectory the guiding sleeve 204 may be locked into the selected alignment by appropriate locking mechanisms, and the operator can insert the needle into the body to obtain the biopsy in any acceptable manner or perform the other procedures with similar longitudinal instruments. For depth control of the needle or instrument, conventional references are used, e.g. calibrated distance marks on the needle that allows the needle to be inserted to a pre-calculated targeted depth in a manner well known in the art.

In one embodiment, the trajectory guide also serves as a needle holder in case control scans are needed to verify the position of the needle in the body. For example, the guiding sleeve 204 may include a mechanism for clamping or otherwise maintaining the needle at a selected depth in the guiding sleeve. Alternatively, the needle may be maintained in place by frictional contact with the patient and the sleeve may simply maintain the needle in the selected trajectory. The trajectory guide also can minimize the risk of the needle bending during insertion by using an elongate tubular member for the guiding sleeve, which can maintain the needle in a straight line at least within the sleeve. A longer guiding sleeve 204 may he used to provide increased bending resistance. Different diameters of guiding sleeves may be provided to accommodate compatibility to various needles thus making the trajectory guide an open platform.

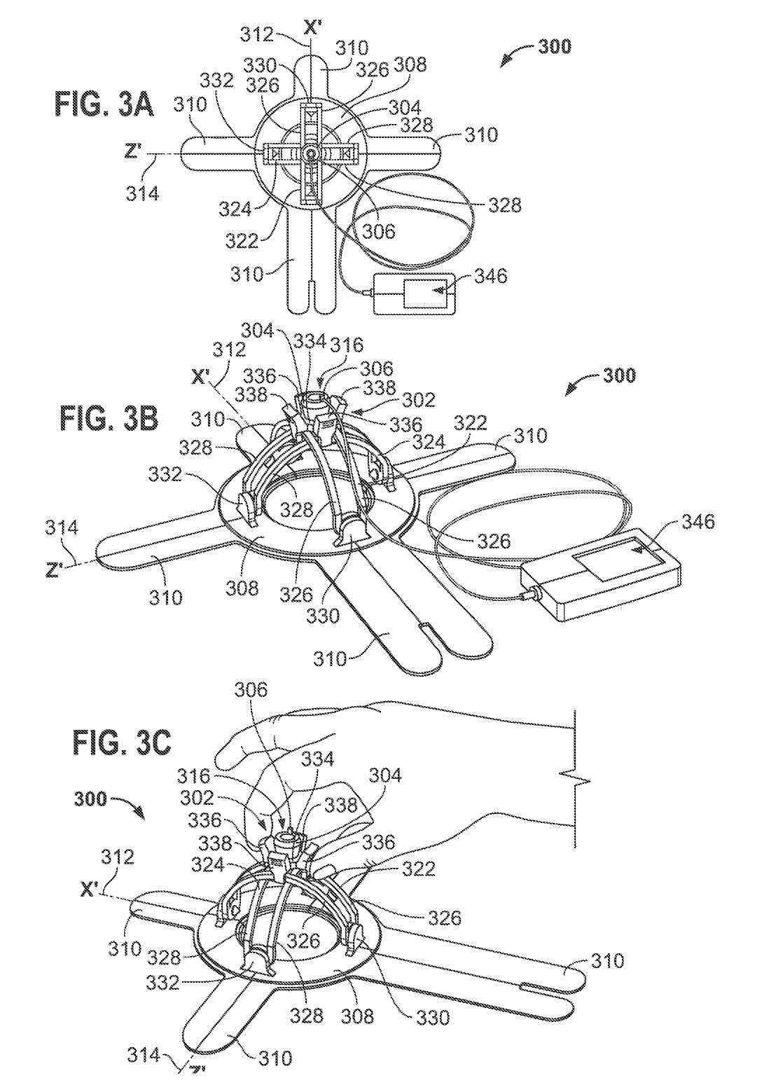

An alternative trajectory guide 300 with a locking mechanism 302 is depicted in FIGS. 3A-3C. The trajectory guide 300 is similar in most respects to the trajectory guide 200, For instance, the trajectory guide 300 includes an axial guiding sleeve 304 with an integrated angle sensor 306 that measures the angle of the guiding sleeve 304 with respect to a local gravity vector. The trajectory guide 300 has an appropriate data interface to transfer data from the sensor 206 to a display screen 346, such as a data port for wires or a wireless transmitter or transceiver, The trajectory guide 300 also includes a base 308, an outer angular guide 322 and inner angular guide 324 that are disposed orthogonally to each other and aligned in the X′-Y′ and Z′-Y′ planes, respectively. A carriage member 334 is slidably guided by each of the angular guides 322 and 324 is attached to the guiding sleeve 304. in addition, one or more visible markers (not shown) may be disposed on the base 308 and aligned with one or both of the X′ 312 and Z′ 314 axes in order to enable alignment of the trajectory guide 300 with the X and/or Z axes 110, 112 of the CT scanner 100 as described previously. Other similarities between the trajectory guide 200 and trajectory guide 300 will be apparent from the figures and numerical identifiers.

Additionally, the trajectory guide 300 includes a locking mechanism 302 associated with the angular guides 236, 238. As depicted in FIGS. 3A-3C, the locking mechanism 302 comprises locking clamps 336 and 338. Each locking clamp 336, 338 includes two clamp members. A middle portion of each clamp member is formed integrally with or otherwise attached to the carriage member 334. The middle portions of the locking clamps 336, 338 serve as a pivot that allows the upper and lower portions of the locking clamps 336, 338 to rotate about the middle portion.

Frictional forces between the locking clamps 336, 338 and the rails 326, 328 prevent the angular guides 322, 324 from moving in the absence of an outside force. The lower portions of clamp 336 are releasably engaged with the rails 328 of inner angular guide 324, and the lower portions of clamp 338 are releasably engaged with the rails 326 of outer angular guide 322. As shown in FIG. 3C, the locking clamps 336, 338 may be released from engagement with the rails 326, 328 by compressing an upper portion of the clamps 336, 338. Compressing or squeezing together the upper portions of the clamps 336, 338 causes the lower portions to pivot outwardly about the middle portion and disengage from the rails 244, 246.

In the embodiment shown, the clamps 336, 338 must be continuously compressed in order to move an angular guide 322, 324. If an operator stops squeezing or compressing the clamps 336, 338, the clamps 336, 338 will reengage with the rails 326, 328. In order to move outer angular guide 322, an operator must compress locking clamp 336. In order to move inner angular guide 324, an operator must compress locking clamp 338. Thus, the locking mechanism 302 is configured to allow separate, independent, movement of each angular guide member 322, 324.

In alternative embodiments, the locking clamps 336, 338 may not have to be held in a continuously compressed state in order to move the angular guides 322, 324. Persons of ordinary skill will recognize that the locking clamps 336, 338 may be configured such that sufficient compression will “snap” the locking clamps 336, 338 out of engagement with the rails 326, 328. Thereafter, an operator may release the locking clamps 336, 338 without the clamps 336, 338 reengaging the rails 326, 328. The upper portions of the locking clamps 336, 338 may be pushed apart, or the lower portions of the locking clamps 336, 338 may be pushed together, in order to “snap” the locking clamps 336, 338 back into engagement with the rails 326, 328. Once the locking clamps 336, 338 are “snapped” back together, the angular guides 322, 324 will once again be prohibited from moving.

Unlike the trajectory guide 200 depicted in FIGS. 2A-2D, the trajectory guide 300 depicted in FIGS. 3A-3C has a circular or annular base 308 rather than a rectangular one. The base 308 may also have a large aperture, though persons of ordinary skill will understand that the size of the aperture may be varied. Because of the size of the aperture, an operator may be afforded a better view of the entry point of an instrument inserted into the guide sleeve 304. Additionally, the annular base 308 has adhesive strips 310 extending outwards from the base 308 aligned with the X′ 312 and Z′ 314 axes. Further, unlike the trajectory guide 200, the trajectory guide 300 does not include servo-motors. The angular guides 322, 326 must be moved by hand in order to accommodate the locking mechanism 302.

The trajectory guide 300 may be more convenient and appropriate for simpler procedures. The operation of the trajectory guide 300 is simple and intuitive. Additionally, with no motors or controller, the trajectory guide 300 may be provided at a lower cost than the trajectory guide 200.

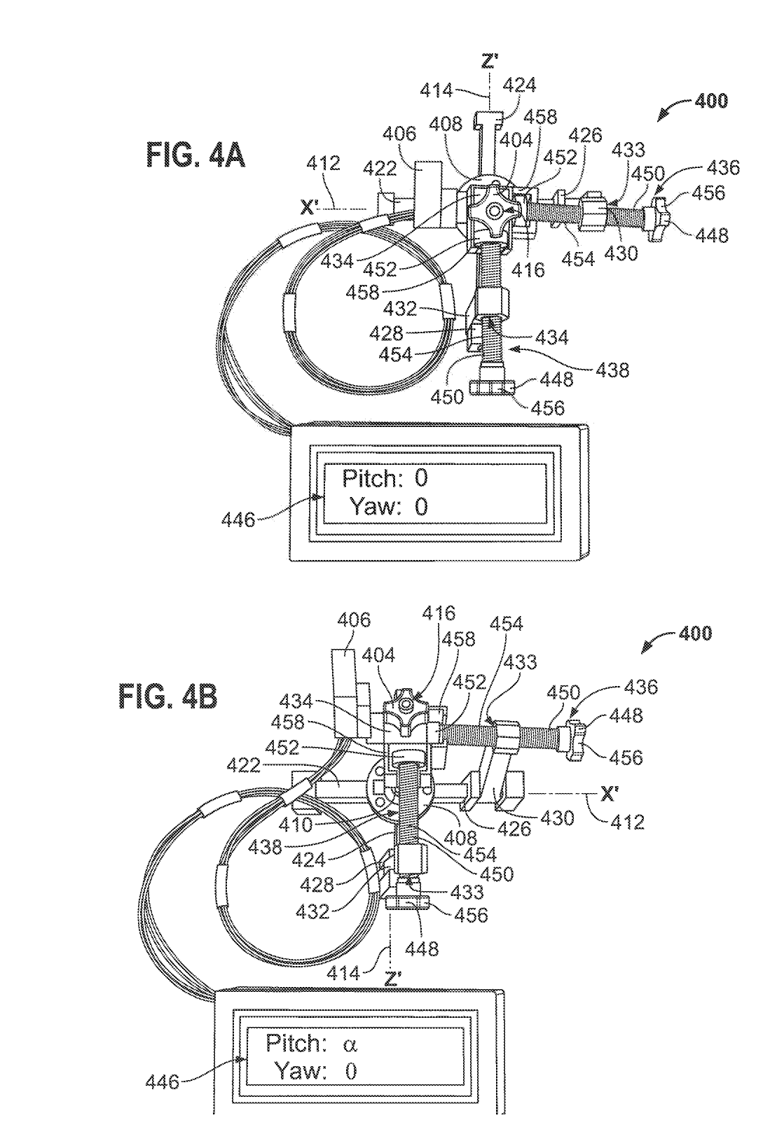

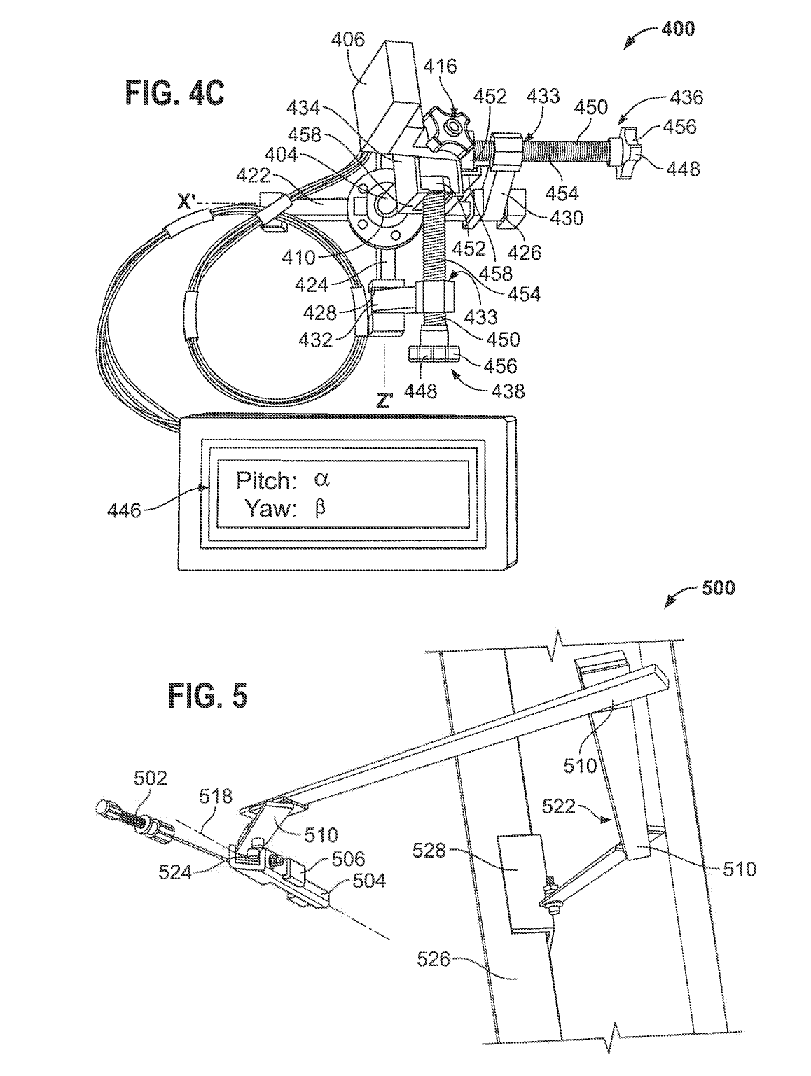

FIGS. 4A-4C show a trajectory guide 400 that comprises another alternative embodiment similar in some respects to the trajectory guide 200. The trajectory guide 400 also includes an axial guiding sleeve 404 that has an integrated angle sensor 406, such as a sensor based on micro-chip MEMS (micro electrical mechanical sensor) technology or an accelerometer that measures the angle of the guiding sleeve 404 with respect to the local gravity vector. The trajectory guide 400 has an appropriate data interface to transfer data from the sensor 206 such as a data port for wires or a wireless transmitter or transceiver, to allow a controller, display, computer, or any other appropriate device.

The guide sleeve 404 is connected to a base 408 configured for attachment to a patient's skin at a predetermined entry point. The guiding sleeve 404 is in the form of an elongate hollow open-ended tube and is attached to the base 408 in a manner that allows the guiding sleeve 404 to move relative to the base. The connection between the base 408 and the guiding sleeve 404 can be any suitable joint or connection such as but not limited to a ball-and-socket joint (not shown). The guiding sleeve 404 is configured to pivot independently about two degrees of freedom through a pivot point 410. A carriage member 434 is attached to the guiding sleeve 404 such that the guiding sleeve 404 is prevented from rotating about its longitudinal axis 418.

Reference may be made to a similar trajectory guide 400 coordinate system that includes an X′-axis 412 and a Z′-axis 414, which extend perpendicular to one another along the base 408 and through the pivot point 410. The pivot point 410 is disposed at or near the middle of the base 408 where the X′-axis 412 and the Z′-axis 414 intersect. The pivot point 410 is in a fixed position with respect to the base 408 and is designed to be placed directly on top of the entry point on the patient such that the pivot point 410 can be substantially coterminous with the entry point during use of the trajectory guide 400. The base 408 is preferably a fiat planar member, and is depicted in FIGS. 4A-4C as disc shaped, However the base may be other shapes capable of being placed against the patient and preferably maintaining the trajectory guide 400 in a stable aligned position. The base 408 has an aperture (not visible) therethrough and encompassing the pivot point 410. The sleeve 404 has an aperture 416 that is sized to allow selected surgical tools, such as a biopsy needle, probe, or other axial device (not shown), to extend through the sleeve, through the base 408 aperture, through the entry point, and into the patient.

The base 408 includes a linear extension 422 aligned with the X′ axis 412 and a linear extension 424 aligned with the Z′ axis 414. The extensions 422, 424 may be continuous or discontinuous through the base 408 to accommodate the aperture in the base 408 and the guide sleeve 404 attachment means. A. pair of support members 426, 428 are pivotally or rotatably attached to extensions 422, 422 at joints 430, 432, Support member 426 is attached to joint 430 such that the support member 426 is pivotable or rotatable about the X′ axis 412. Support member 426 is attached to joint 430 such that the support member 426 is pivotable or rotatable about the Z′ axis 424. In a neutral position, the support members 426, 428 extend upwardly from the extension 422, 424, approximately parallel to the guide sleeve 404.

Both support members 422, 424 have an aperture 433 at an end opposite the respective joint 430, 432. The apertures 433 may be circular, as depicted in FIGS. 4A-4C, or some other suitable shape. The portions of the support members 430, 432 surrounding the respective apertures have grooves configured to engage with screw threads 454 of linear drives 436, 438.

Each linear drive 436, 438 includes a knob 448, a rod-shaped shaft 450, and a connecting end 452. Each shaft 450 has screw threads 454 configured for engagement with appropriate support member 430, 432. Each knob 448 is connected to an end of each shaft 450 such that turning the knob 450 results in turning of the shaft 450 and associated screw threads 454. The knobs 448 have ergonomic grips 456 to assist in gripping and turning by an operator or user. Additionally, grips 456 may be helpful in a motorized version of trajectory guide 400.

The connecting end 452 of each linear drive 436, 438 is connected to carriage member 434. Carriage member 434 includes grooves 458 to accommodate each connecting end 452. At a neutral position, the grooves 458 are approximately aligned with apertures 433. As depicted in FIGS. 4A-4C, each connecting end 452 may be circular or disc shaped, and grooves 458 may be rectangular and dimensioned so as to snugly accommodate each connecting end 452 therein. Alternatively, grooves 458 may be circular and each connecting end 452 may be rectangular, or both grooves 458 and connecting ends 452 may be rectangular or circular. In a further alternative embodiment, grooves 458 and connecting ends 452 may be triangular, helical, spherical, an alternative polygon and/or any other suitable shape.

In use, an operator or user may position the trajectory guide 400 at an appropriate position on a patient. The base of the trajectory guide 400 may be treated with adhesive or otherwise configured to securely attach to a patient. The trajectory guide 400 may initially be at a neutral orientation such as depicted for example in FIG. 4A. When at a neutral orientation, the display screen 446 shows the pitch and yaw angles of the guide sleeve 404 to be 0. In addition, support members 430, 432 are substantially perpendicular to extensions 422, 424, and substantially parallel to guide sleeve 404. Further, substantially equal portions of drive member 436, 438 shafts 450 are disposed on either side of the appropriate support member 430, 432. In order to change the orientation of the guide sleeve 404, an operator may adjust one or both of the drive members 436, 438 by rotating the appropriate knob 448. Turning the knob 448 causes the screw threads 454 of the shaft 450 to turn in the grooves of the appropriate support member 430, 432, and causes the shaft 450 to move through the support member 430, 432 toward or away from the guide sleeve 404. Turning the knob 448 in one direction will move more of the shaft 450 towards the guide sleeve 404, and move the knob 448 itself towards the support member 430, 432. As the shaft 450 moves towards the guide sleeve 404, the connecting member 452 will push on the carriage member 434, causing the guide sleeve 404 to tilt about the base 408. Turning the knob 448 in the other direction will move more of the shaft away from the guide sleeve 404, and move the knob away from the appropriate support member 430, 432 as well. As the shaft 450 and knob 456 move away from the guide sleeve 404 the connecting end 452 pulls on the carriage member 434, causing the guide sleeve 404 to tilt. As drive member 436 pushes or pulls on the carriage member 434, causing the guide sleeve 404 to tilt, support member 432 rotates about its joint 428 to accommodate the tilt of the guide sleeve 404, the carriage member 434, and the resulting movement of its drive member 438 which is attached to the carriage member 434. Similarly, as drive member 438 pushes or pulls on the carriage member 434, support member 430 rotates about its joint 426 to accommodate the tilt of the guide sleeve 404, carriage member 434, and its own drive member 436. Preferably the knob 448 is sized such that it cannot go through the support member 430, 432, thereby creating a limit to the extent to which the shaft 450 and the knob 448 may be moved towards the guide sleeve 404. Likewise, the connecting member 452 and the carriage member 434 are sized such that they may not be moved through the appropriate support member 430, 432, thereby creating a limit to the extent to which the shaft 450 and the knob 448 may be moved away from the guide sleeve 404, Thus, the trajectory guide 400 is configured such that the drive members 436, 438 may be independently and separately adjusted.

As shown in FIGS. 4A-4C, display screen 446 shows the pitch and yaw angles of the guide sleeve 404, as reported by the angle sensor 406. In a preferred embodiment, the angle sensor 406 communicates the pitch and yaw angles with respect to a local gravity vector, as described above. FIG. 4A depicts the trajectory guide 400 at a neutral orientation, where the pitch and yaw angles are both 0. FIG. 4B depicts the trajectory guide 400 where the yaw angle is 0 and the drive member 438 has been adjusted to the point where the pitch angle is a non-zero value α. FIG. 4C depicts the trajectory guide 400 where the drive members 436, 438 have been adjusted to the point where the pitch angle is a non-zero value α and the yaw angle is a non-zero value β.

In an alternative embodiment of the trajectory guide 400, a motor (not shown) may be included to automate the adjustment of the linear drives 436, 438. In such an embodiment, a motor or a plurality of motors may be configured to turn the knob 448 or shaft 450 of one or more of the linear drives 436, 438 in order to adjust the angle of the guide sleeve 404. Further, display 446 may be configured to be a controller with input means for a user to indicate the desired guide sleeve 404 angles, similar to the controller described with respect to the trajectory guide 200. Alternatively, the controller may include an input means that is configured to allow a user to indicate the desired displacement of the linear drives 436, 438. The motor(s) may be configured to adjust the linear drives 436, 438 in response to controller input from a user.

Turning to FIG. 5, another alternative trajectory guide 500 is shown. The alternative trajectory guide 500 includes a guiding sleeve 504 similar to those previously described, configured to guide a medical instrument, such as a biopsy needle 502, and allow passage (at least part way) therethrough. In order to prevent the guiding sleeve 504 from rotation about its longitudinal axis 518, a linking mechanism 522 is attached to a distal end 524 of the guiding sleeve 504 and to a separate fixture 526, such as a patient table, at an attachment point 528. The linking mechanism 522 may be attached to the fixture 526 by any suitable means, including, but not limited to, clamps, adhesive, screws, bolts, welding, friction, rails, and/or any combination of the aforementioned. The attachment point 528 can be moved along the fixture 526 such that the axis of the guide is aligned with the entry point along the laser line (X- or Z-axis of the CT scanner). The linking mechanism 522 includes one or more pivot joints 510 sufficient to allow the guiding sleeve 504 to be pivoted in two degrees of freedom, i.e., pitch and yaw, about the attachment point in a manner clear to a person of skill in the art. The trajectory guide 500 includes one or more angle sensors 506 fixedly associated with the guiding sleeve 504 in any manner as previously described herein sufficient to provide automatic pitch and yaw angle measurements in relation to gravity and an appropriate data interface sufficient for providing the angle measurements to an external computer, display, controller, and/or console. Appropriate locking mechanisms may be used to secure the linking mechanism 522 and guiding sleeve 504 in a selected trajectory in a manner clear to a person of skill in the art.

The trajectory guide 500 is useful for situations where it is not possible to easily attach or secure a trajectory guide base to a patient around the entry point, for example, in cases of breast biopsies. This may be because the skin surface around the planned entry point is not well suited to holding a trajectory guide due to high curvature or a consistency that is too flabby, or a number of other reasons. Additionally, the trajectory guide 500 may provide an operator a better view of the entry point of an instrument inserted into the guide sleeve 504 because there is no base to obstruct the view.

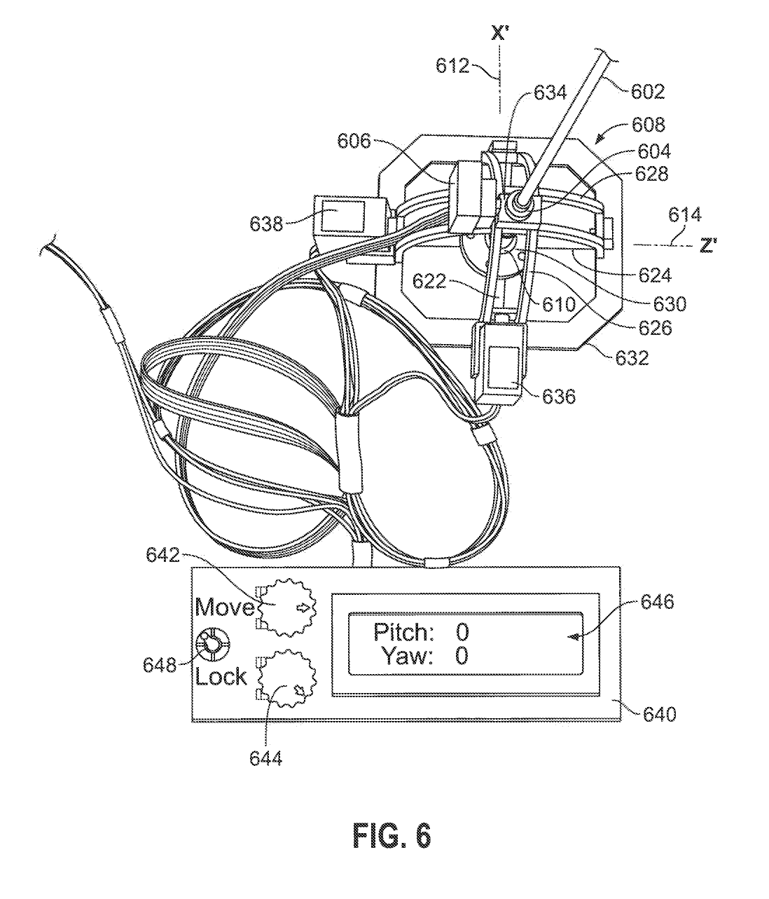

Turning to FIG. 6, yet another alternative trajectory guide 600 is shown. The trajectory guide 600 is similar to other trajectory guides mentioned above, as will be apparent from its depiction and similar numbering.

The trajectory guide 600 includes a base 608 with a disc shaped interior section 630 and extensions 622, 624 that branch out from the interior section 630 aligned with X′ 612 and Z′ 614 axes, similar to the trajectory guide 400. The base 608 further includes a hexagon shaped outer section 632 that surrounds the interior section 630 and intersects with the extensions 622, 624.

The trajectory guide 600 includes a guide sleeve 604, angle sensor 606, carriage member 634, angular guides 626, 628, motors 636, 638, and controller 640, similar to those described above with respect to the trajectory guide 200. The controller 640 includes input knobs 642, 644 for an operator/user to input desired pitch and yaw angles of the guide sleeve 604. Additionally, the trajectory guide 600 controller 640 includes a switch 648. The switch 648 has two positions: move, and lock. When the switch 648 is in the “move” position, the controller 640 sends a signal to indicate that the motors 636, 638 should begin moving the angular guides 626, 628. In such an embodiment, the angular guides 626, 628 are not moved by the motors 636, 638 until the switch is toggled to the “move” side. After the switch is toggled to the “move” side, the motors adjust the angular guides 626, 628 until the actual angles measured by the angle sensor 606 corresponded to the angle values inputted by the operator. When the switch 648 is in the “lock” position, the controller 640 will not send a signal to the motors 636, 638 indicating that the motors 636, 638 should begin moving the angular guides 626, 628. Thus, any input to the controller 640 by way of the knobs 642, 644 will effectively be ignored. This can prevent unintentional modification of the pitch and yaw angles of the guide sleeve 604 through accidental or erroneous movement of the knobs 642, 644. The operation of the trajectory guide 600 is otherwise similar in most relevant respects to that which is described above with respect to the trajectory guide 200.

As mentioned before, in some embodiments, it may be important to prohibit movement of the guiding sleeve when secured to the patient in degrees of freedom that cannot be measured by the sensors. Thus, it can be seen that in all of the described solutions, rotation around the Y-axis, i.e., by rotation about the longitudinal axis of the guiding sleeve, is prohibited. In other words, by prohibiting rotation in the “roll” direction and maintaining the alignment of the Z-axes of all involved components, it is possible for pitch and yaw to become absolute measures that define the trajectory unambiguously.

However, in other embodiments it may not be important to prohibit movement of the guiding sleeve. In such an embodiment, the guide sleeve may be allowed to rotate about a longitudinal axis of the guide sleeve while still maintaining alignment with the CT or the patient coordinate system. Further, an angle sensor may be positioned independently of the guide sleeve and/or trajectory guide such that the rotation of the guide sleeve does not affect the angle sensor. Alternatively, the angle sensor may be attached to some other part of the trajectory guide rather than the guide sleeve such that the rotation of the guide sleeve does not affect the angle sensor. In a further alternative, a position indicating device like a video camera providing feedback about the position and orientation of the guide sleeve may be used instead of an angle sensor

### INDUSTRIAL APPLICABILITY

The axial surgical trajectory guides of the present invention are useful for setting a trajectory of a needle for obtaining a biopsy based on CT images and other surgical procedures that require guidance of an axial surgical instrument where the position of the patient relative to the local gravity vector is known or calculated. The axial surgical trajectory guides of the present invention can overcome the need for a separate surgical navigation system, such as an optical or magnetic system, that would require registration of the CT image with the data from the navigation system. Further, the axial surgical trajectory guides of the present invention eliminate the need to maintain the guide in a pre-defined relationship with respect to the horizontal plane or gravity vector. Thus, the axial surgical trajectory guides of the present invention provide a significant benefit by reducing the complexity of equipment necessary in the procedure or operating room and/or simplifying the actual procedure of aligning the guide by the operator while defining the selected trajectory in relation to the patient.

Although the trajectory guides herein are described with reference to use with CT scanners, it is clear that the trajectory guides may be used with other systems in which a coordinate system of a three-dimensional image of a patient can be physically transferred directly to the coordinate system of the trajectory guides by, for example, laser lines or other visible means, consistent with the principles disclosed herein.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved. All patents, patent applications, and other printed publications identified in this foregoing are incorporated by reference in their entireties herein.

I/We claim:

1. A medical trajectory guide, comprising:
a base configured for alignment with a coordinate axis;
a first guide member rotatably attached to the base, wherein the first guide member is rotatable about a first axis;
a guide sleeve guided by the first guide member; and
a motor coupled to the first guide member and configured to rotate the first guide member in relation to the first axis.

2. The medical trajectory guide of claim 1, wherein the guide sleeve has a longitudinal axis, and wherein the guide sleeve is prevented from rotating about its longitudinal axis.

3. The medical trajectory guide of claim 1, further comprising an electronic angle sensor configured to sense an angle of the guide sleeve relative to a first plane defined by the first axis and a gravity vector.

4. The medical trajectory guide of claim 1, further comprising a controller in communication with the motor, wherein the motor is configured to rotate the first guide member about the first axis according to input from the controller.

5. The medical trajectory guide of claim 3, further comprising a display unit in communication with the electronic angle sensor, wherein the display unit is configured to display the angle of the guide sleeve relative to the first plane.

6. The medical trajectory guide of claim 4, wherein the motor is configured to automatically rotate the first guide member to maintain the angle of the first guide member in relation to the first plane if and when the first plane is moved.

7. The medical trajectory guide of claim 1, further comprising a second guide member rotatably connected to the base, wherein the second guide member is rotatable about a second axis.

8. The medical trajectory guide of claim 7, further comprising an electronic angle sensor configured to sense an angle of the guide sleeve relative to a first plane defined by the first axis and a gravity vector, and wherein the electronic angle sensor is further configured to sense an angle of the guide sleeve relative to a second plane defined by the second axis and a gravity vector.

9. The medical trajectory guide of claim 8, wherein the electronic angle sensor is attached to the guide sleeve.

10. The medical trajectory guide of claim 1, wherein the base includes a mark that visually identifies the first axis so as to allow the first axis to be aligned with a coordinate system of an imaging device.

11. A medical trajectory guide, comprising:
a base configured for alignment with a coordinate axis;
a first drive member support including a first end and a second end, wherein the first end is rotatably attached to the base and the second end includes an aperture;
a guide sleeve including a first end and a second end, wherein the first end is in contact with the base;
a first drive member linearly movable through the aperture of the first drive member support and attached to the second end of the guide sleeve such that movement of the first drive member causes the guide sleeve to tilt; and
a motor configured to move the first drive member through the aperture of the first drive member support.

12. The medical trajectory guide of claim 11, wherein the guide sleeve has a longitudinal axis, and wherein the guide sleeve is prevented from rotating about its longitudinal axis.

13. The medical trajectory guide of claim 11, further including an electronic angle sensor configured to sense an angle of the guide sleeve relative to a local gravity vector.

14. The medical trajectory guide of claim 13, further including a display unit in communication with the electronic angle sensor, wherein the display unit is configured to display the angle of the guide sleeve relative to the local gravity vector as sensed by the electronic angle sensor.

15. The medical trajectory guide of claim 11, further comprising:
a second drive member support including a first end and a second end, wherein the first end is rotatably attached to the base and the second end includes an aperture; and
a second drive member linearly movable through the aperture of the second drive member support and attached to the second end of the guide sleeve such that movement of the second drive member causes the guide sleeve to tilt.

16. The medical trajectory guide of claim 11, wherein the first drive member includes screw threads that are configured to engage with grooves in the second end of the first drive member support.

17. The medical trajectory guide of claim 15, wherein the first drive member includes screw threads that are configured to engage with grooves in the second end of the first drive member support, and wherein the second drive member includes screw threads that are configured to engage with grooves in the second end of the second drive member support.

18. The medical trajectory guide of claim 17, further including a motor configured to move the second drive member through the aperture of the second drive member support.

19. A medical trajectory guide, comprising:
a base configured for alignment with a coordinate axis;
a first guide member rotatably attached to the base, wherein the first guide member is rotatable about a first axis;
a guide sleeve guided by the first guide member;
a first motor coupled to the first guide member and configured to rotate the first guide member in relation to the first axis; and
a controller in communication with the motor, wherein the controller includes a switch having a first position, and wherein the motor is configured to rotate the first guide member about the first axis according to input from the controller if and only if the switch is in the first position.

20. The medical trajectory guide of claim 19, wherein the guide sleeve has a longitudinal axis, wherein the guide sleeve is prevented from rotating about its longitudinal axis, and wherein the medical trajectory guide further includes an electronic angle sensor configured to sense an angle of the guide sleeve relative to a first plane defined by the first axis and a gravity vector.

21. The medical trajectory guide of claim 19, further including a display unit in communication with the electronic angle sensor, wherein the display unit is configured to display the angle of the guide sleeve relative to the first plane.

22. The medical trajectory guide of claim 19, further comprising a second guide member rotatably connected to the base, wherein the second guide member is rotatable about a second axis; and a second motor coupled to the second guide member and configured to rotate the second guide member in relation to the second axis.
